(19) United States
(12) Patent Application Publication
(10) Pub. No.: US 2026/0232871 A1
(43) Pub. Date: Aug. 13, 2026

(54) Coronary Artery Bypass Graft

(71) Applicant: Vascudyne, Inc., St. Paul, MN (US)

(72) Inventor: Zeeshan Syedain, Stillwater, MN (US)

(21) Appl. No.: 19/157,440

(22) PCT Filed: Feb. 21, 2024
(86) PCT No.: PCT/US2024/016722

§ 371 Date: Aug. 18, 2025

Related U.S. Application Data
(60) Provisional application No. 63/447,306, filed on Feb. 21, 2023.

(51) Int. Cl. A61L 27/54; A61F 2/06; A61L 27/36

(52) U.S. Cl. CPC A61L 27/54; A61F 2/06; A61L 27/36; A61L 2430/20

(57) ABSTRACT

The present invention is a coronary artery bypass graft (CABG) formed from a constructed tissue that can be implanted.

The present application claims the benefit of priority to U.S. Application No. 63/447,306 filed 21 Feb. 2023 entitled “Coronary Artery Bypass Conduit.” This document is incorporated herein by reference in its entirety.

### FIELD OF THE INVENTION

The invention relates to products formed from proprietary regenerative tissue, products made from the tissue, an implant comprising this tissue, and methods of treating conditions and/or disorders using such tissue.

### I. BACKGROUND OF THE INVENTION

A relatively new field of medicine—since the early 1990s—is the field of Regenerative Medicine. Regenerative Medicine is the process of creating functional tissues to repair, replace, or restore tissue or organ structure and function lost due to age, disease, damage, or congenital defects. This field of medicine uses new methods and products including tissue engineering or constructed tissue (CT).

The use of prepared heterogenous graft material for human surgical implantation is well known. More specifically, the use of treated animal tissue as human tissue grafts, replacement valves, and similar implantation surgical procedures is well known. However, problems of immunogenicity, thrombogenicity, calcification, material strength, and size have not been adequately addressed in the prior art.

Since the 1930's, medical researchers have attempted to develop suitable natural and synthetic alternatives for obtaining small diameter grafts useful in vascular surgery. Historically, attempts to fabricate such tubular grafts from man-made materials have been somewhat unsuccessful. Homologous tissues are not always readily available and are not always readily available in the size the surgeon needs. Furthermore, some of these tissues may be immunogenic and therefore may require processing or certain treatments to reduce their immunogenicity.

Coronary artery bypass grafts (CABG) remain the mainstay of revascularization for multivessel coronary artery disease (CAD) (2). The most widely used conduits are autologous internal thoracic arteries, radial arteries and saphenous veins, which provide excellent mechanical stability and natural anti-thrombogenicity (2). Patients currently undergoing coronary revascularization surgery present with an increasing age and risk profile (2). In patients with comorbidity of other vascular diseases, the quality of saphenous vein grafts may be impaired if they are available at all (2, 3). Therefore, there continues to be a need for alternative bypass conduits for the coronary revascularization surgery (4-6). Further, even in patients with suitable veins, the procedure to harvest, ensuring all branches are tied and lumen diameter matches to native coronary, is associated with additional complications.

Several clinical studies have evaluated non-autologous grafts for coronary artery bypass graft (CABG) surgery with limited success (4-6). All of these non-autologous materials were either animal-derived or cadaver-based, both of which have poor regenerative capacity and ability to endothelialize. The new approach of tissue engineering a blood vessel from cells in a laboratory, decellularizing and using the cell produced extracellular matrix as a conduit has been shown to regenerate and remodel both in preclinical non-human primate studies (7, 8) and clinical studies (9-13).

Humacyte Inc has pioneered one of these approaches of tissue engineering a vascular graft by seeding vascular cells on a synthetic biodegradable polymer, subsequently culturing it in a bioreactor to produce final acellular ECM tube (8). Humacyte has implanted their vascular conduits in patients needing vascular grafts for dialysis, for femoral artery and for trauma injuries (9-11, 13, 14). In the above knee femoral-to-popliteal artery implant in 20 patients, 6 mm internal diameter conduits of 35-42 cm length were followed up to 24 months. Primary and secondary patency was 58% and 74% respectively. Further, immunological panel testing showed no change in panel reactive antibodies after implant. The most common cause for loss of primary patency was thrombosis and anastomosis stenosis (10). Humacyte recently reported the outcomes using the conduits for coronary artery bypass in a non-human primate model (AHA Conference 2022).

A variety of materials are available, but all have shortcomings, and none are regenerative. There remains a need for a solution that combines the benefits of native collagenous tissue having regenerative properties with those of manufacturing at scale and in the proper form (i.e., conduit).

Vascular graft failures are commonly associated with thrombosis, intimal hyperplasia, atherosclerosis, and/or infection. Implanted grafts of the present invention show no signs of any of these failure signs.

Notwithstanding the usefulness of the above-described methods, a need still exists for increasing patency; making an implant less thrombotic in structure and/or function; minimizing calcification; and increasing the useful life of the implant.

### II. SUMMARY OF THE INVENTION

One embodiment of the invention is the preparation and use of constructed tissue (CT) to make implants, their use as implants, and their use in mediating treatment or therapy. In preferred embodiments of the invention, the implant is a coronary artery bypass graft (CABG), a peripheral vascular graft, or a saphenous vein conduit.

The present invention utilizes a novel tissue engineering approach that uses human fibroblasts seeded in a biological polymer followed by bioreactor culture leading to a final acellular ECM vascular conduit that is completely biological with mechanical properties comparable to the internal mammary artery (7, 15). The tissue may be characterized in part by beneficial collagen alignment; anisotropy; anisotropy in vivo; acellular; compatible with terminal sterilization; non-chemically crosslinked; and typical moisture content about 85%. In preferred embodiments of the invention, the tissue includes non-uniform collagen layers, typically, a high-density collagen layer, then a porous layer, then a high-density layer, then another porous layer. In some embodiments, the non-uniform collagen layer comprises a high-density layer sandwiched between two or more porous layers. The inventors believe that these non-uniform collagen layers provide many advantages, including but not limited to providing more strength to the finished tissue, e.g., greater burst strength. The vascular conduit (6 mm diameter conduit) has been implanted in 16 patients in two trials to date for use in hemodialysis access (12). In the first-in-human, 6-month study showed favorable patency and low complication rate establishing the initial safety and feasibility of the tissue's use for dialysis access in patients with end-stage kidney disease. The mechanical durability and lack of immune response established the tissue as a potential regenerative material for clinical use in dialysis and other vascular applications.

An advantage of both embodiments of the invention is the starting material itself.

The tissue of the present invention is a completely biological, acellular collagenous matrix produced by allogeneic, neonatal, human dermal fibroblasts (nHDF). The nHDF are mixed with thrombin and fibrinogen and molded into the desired shape. The molded structure is then cultured in a bioreactor where the nHDF degrades the fibrin gel and secretes collagen and other extracellular matrix (ECM). The resulting tissue is then decellularized, yielding an acellular human collagenous matrix.

The products, uses, and processes of the present invention are suitable for treating diseases and conditions that would benefit from regenerative engineered tissues, especially those involving tubular tissue constructs. One such use is for coronary artery bypass grafts (CABG).

Some embodiments of the invention include the manufacture of a graft that is durable and has been demonstrated in both animal and human trials to withstand the rigorous mechanical requirements of the vascular system (8).

The biological materials according to the present invention, are processed to modify (e.g., reduce or eliminate) size and shape, thinness, collagen content, and other characteristics and properties that will become clear from the description of the invention. The methods, uses, and products of the present invention are intended for implant in a mammal, preferably a human. All of the biological materials, processed according to the present invention, are appropriate for use in an in vivo environment, and include one or more of the following desirable properties for graft material suitable for implantation: a) size compatibility with surrounding vessels to which it will be anastomosed; b) sutureability, kink resistance, softness, radial and longitudinal compliance, and flexibility (a softer hand); c) non-thrombogenicity or low levels of thrombogenicity, particularly after regeneration or recellularization; d) durability; e) ease of sterilization; f) readily available, and available in diameters and lengths appropriate for surgical procedures; g) shelf life appropriate for market conditions (typically greater than three years); h) resistant to infection; i) sufficient strength to resist aneurysm formation; j) non-immunogenic; k) resistant to degradation; l) resistant to formation of neointimal hyperplasia; m) tactile, as expressed by surgeons using the tissue and/or grafts of the present invention, particularly suitable for microsurgery; n) is anti-bacterial; o) is non-infectious; p) is anti-microbial; and q) under circumstances when the implant becomes infected, the implant of the present invention may not become infected or resists infection (e.g., the host/recipient cells become infected exclusive of the implant); r) reduced or eliminated calcification characteristics; s) improved or biologically appropriate or desirable resorbability; t) becomes or transitions into living tissue; and u) in some embodiments, capable of endothelialization. As used herein, living tissue refers to a tissue that exhibits the presence of active cells (originating from the implant recipient), e.g., including but not limited to producing elastin, producing ECM; and/or an un-paralleled lack of immunogenicity.

Surgeons have also recognized the superior quality of the tissue of the present invention in terms of a tactile response, specifically in comparison to typical vasculature (usually autologous, e.g., venous grafts during coronary artery bypass procedures).

A graft of the present invention provides several other advantages: the graft allows revascularization of the diseased heart using a tissue that exhibits little or no discernable immune response; isodiametrically uniform, which contributes to excellent laminar flow within the graft, which in turn reduces turbulence and turbulence related thrombus formation; a graft that grows with the body, making this graft useful and beneficial for pediatric patients; the graft eliminates the need for vein and artery harvesting procedures, thereby eliminating the need for a second surgery; provides a small diameter, consistently uniform sized graft; and provides an excellent graft with high patency rates and mechanical characteristics.

Although specific advantages have been enumerated above, various embodiments may include some, none, or all of the enumerated advantages.

Other technical advantages may become readily apparent to one of ordinary skill in the art after review of the following FIGURES and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

With the following enabling description of the drawings, the apparatus should become evident to a person of ordinary skill in the art.

### III. BRIEF DESCRIPTION OF THE FIGURES

FIG. 1 is a chart showing the primary and secondary patency of a tissue of the present invention.

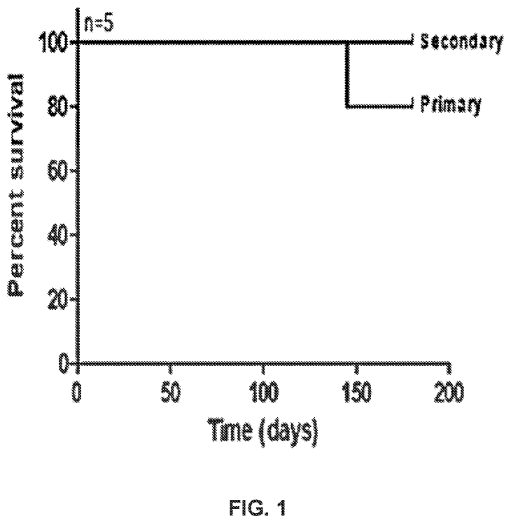

### IV. DETAILED DESCRIPTION OF THE INVENTION

The present invention is a coronary artery bypass graft (CABG) formed from constructed tissue (CT) produced according to the present invention. The graft of the present invention may be used as a vascular conduit anywhere it might be beneficial in vivo in a mammal, including a human. Exemplary uses include but are not limited to CABG surgery, preferably for bypassing a blocked or damaged portion of any coronary artery, for restoring blood flow to the heart; a peripheral vascular graft; or a saphenous (superficial or deep) vein conduit, including but limited to above the knee, around the knee, or below the knee.

In preferred embodiments of the invention, the tissue or graft is used to bypass the left anterior descending artery (LAD) coronary artery as the primary target. In the most preferred embodiments, the graft or implant is between the left internal mammary artery (LIMA) to the LAD. In some embodiments of the invention, the tissue is used as a bypass to a second or third target (non-LAD coronary artery) in patients needing coronary artery bypass grafting (CABG). The implant is a tubular bypass conduit that creates a new path for blood to flow around a blocked or partially blocked artery in the heart, improving blood flow to the heart muscle.

As noted above, a wrap or tissue product of the present invention is a tube or conduit, or tubular is shape. A preferred embodiment is a continuous tubular shape. Typically, these shapes include or enclose a lumen through the longitudinal length of the construct. The lumen can be open or closed at one or both ends.

The present invention is a product and process for use in any in vivo treatment in which endothelization and recellularization provides a beneficial result for the patient/recipient.

As used herein, recellularization refers to the repopulation or growth of cells and structures near the implant site aiming to reconstitute and recreate the natural tissue-specific function. In some embodiments of the invention recellularization includes the tissue growing, including somatic growth, with the patient.

As used herein, healing refers to the ability of living organisms to replace damaged or lost tissue with new cells, heal damaged tissue, restoring their structure and function, that is, becoming living tissue. Healing includes repopulation leading to restoration of tissue and/or body functions, including but not limited to restoration of cells, other biological molecules, and biological structures, including acellular ECM scaffolds. Healing is the process of renewal, regrowth, or restoration of a tissue, organ, or organism after damage, injury, or disease.

As used herein, remodeling refers to the process by which the body adapts to and integrates an implanted medical device. This process involves the interaction between the implant and the surrounding tissues, which can lead to changes in the structure and composition of the tissue, and in healing.

As used herein, recellularization and remodeling are considered to be within the definition of regeneration.

In the most preferred embodiments, the tissue of the present invention, when implanted, does not mechanically weaken during the healing process. One of the benefits of a tissue of the present invention is that it does not need a scaffold or the like. This feature is in contrast to products that have a synthetic or biological portion that is specifically degradable, e.g., a scaffold. This feature is also distinct from the tissue as it is being manufactured. During formation, the ECM-producing cells degrade fibrin until a collagenous tissue is formed. The cell-containing tissue contains no, very little, or not detectable amounts of fibrin.

An embodiment of the invention includes but is not limited to a tissue of the present invention that recellularizes without first degrading.

Embodiments of the invention include but are not limited to the form of tissue delivery. Examples include endovascular delivery and surgical implant.

A tube or tubular material of the present invention may be positioned to replace, surround, contain, or enclose a body structure. Examples include but are not limited to one or more coronary arteries.

The graft or implant of the present invention may be used in a method of treating a patient having a wide variety of conditions, diseases, or injuries, the common theme of which is that the treatment involves a tissue implant. Exemplary diseases and conditions include but are not limited to arrythmia, wound infections, kidney/renal failure, thrombosis/embolization, aneurysm, patient infection, and patient immune response.

In another embodiment of the invention, the tissue may include structures or agents (e.g., active agents) within the surface of the CT tissue.

The tissue of the present invention may be any size or shape. In a preferred embodiment, the tissue is the form of a wrap, conduit, sheet, cover, envelope, or tube.

The present invention is a graft, prosthesis, or covering formed from constructed (CT) regenerative, and/or engineered tissue. As used herein, constructed or engineered refers to the fact that the inventors and others may produce or construct the tissue, e.g., the tissue is not a product of nature. In preferred embodiments of the invention, the tissue mediates regeneration without causing degradation of tissue and other biological material in the area of the implant site. The invention includes methods of making the tissue and methods of making the graft or prosthesis.

The tissue may be formed by combining ECM-producing cells in the presence of fibrinogen and thrombin under conditions that permit the formation of regenerative tissue. Typically, the process involves forming a cell-seeded suspension comprising ECM producing cells, fibrinogen, and thrombin. The suspension is then cast over a form and allowed to incubate. During incubation, an ECM/fibrin/collagen tissue begins to form. As part of that process, compaction and fiber alignment may occur, leading to remodeling of the ECM/collagen/fibrin tissue as it forms. The tissue is then cultured until it matures, e.g., is substantial enough to be used for its intended purpose. The resulting cell-containing tissue is the decellularized. The tissue of the present invention is cultured from completely biological raw materials and allogeneic dermal cells. For example, see the patents and patent applications listed below.

In preferred embodiments of the invention, the hydrogel—ingredients in a suspension—allows the tissue to grow in a volumetric 3-D process also known as casting. In many ways, the process from initial ingredients to a complete tissue represents a remodeling continuum. In contrast, most if not all typical tissue engineering methods use a synthetic and/or immunogenic scaffold or the like to grow the tissue in a 2-D manner (cell suspension seeded on the surface). The growth eventually produces a 3-D construct, but the growth in scaffold-based constructs is different that the volumetric 3-D casting growth in tissues of the present invention. A preferred embodiment of the invention is any structure or shape formed from the tissue of the present invention, including but not limited to a tubular graft.

In accordance with embodiments of the present invention, any prosthesis may be formed in whole or in part using regenerative tissue (RT) or engineered tissue. RT, as used herein, refers to tissue formed or processed as disclosed in the following: 2007/061800; WO 2007/092902; 2016/0203262; WO/2004/018008; WO 2004/101012; PCT/US21/62709 (filed 9 Dec. 2021); PCT/US2017/026204 (filed 5 Apr. 2017); U.S. Pat. Nos. 10,111,740; 10,105,208; 10,893,928; 8,192,981; 8,399,243; 8,617,237; 8,636,793; 9,034,333; 9,126,199; U.S. Ser. No. 17/139,575 filed Dec. 31, 2020 (issue fee paid); U.S. Ser. No. 16/500,147 filed Oct. 2, 2019 (issue fee paid); U.S. Ser. No. 10/523,618; U.S. Ser. No. 10/556,959; U.S. Ser. No. 13/771,676; 2015/0012083; 2009/0319003; 2011/0020271; 2012/0230950; 2013/0013083; 2014/0330377; 2014/035805; 2017/0135805; 2017/0296323; 2017/0306292; U.S. Pat. Nos. 8,198,245; 9,127,242; 9,556,414; 9,657,265; and 9,650,603; all of which are hereby incorporated in the entirety be reference.

In one embodiment of the invention, the bioengineered tissue may be made according to U.S. Pat. Nos. 10,111,740; 10,105,208; 10,893,928; and 11,589,982, all Tranquillo, et al., each incorporated in its entirety be reference. Any process or method for producing engineered tissue involving ECM-producing cells in a hydrogel is included within the scope of the present invention.

The CT of the present invention, may be characterized by lack of evidence of patient infection (in vivo); lack of evidence of patient immune response (in vivo); lack of evidence of toxicity; lack of evidence of implanted tissue degradation; lack of evidence of residual cellular debris (e.g., particle shedding from the tissue, in contrast to polymer degradation and erosion); modified (e.g., reduce or eliminate) inflammation, calcification characteristics, resorbability, resorption, absorption, suture retention, size and shape, thinness (e.g. dilatation or aneurysm formation), collagen content, and other characteristics and properties that will become clear from the description of the invention.

The CT of the present invention is distinct from certain other kinds of constructed tissue in the use of completely biological raw materials and allogeneic dermal cells; and in the use of crosslinked fibrinogen that is later degraded during the culturing process. Also, the CT of the present invention can be contracted or allowed to contract, for example, in the longitudinal direction and/or in the radial direction, among others. In accordance with some embodiments of the invention, the fibers in the tissue may align or become aligned, believed to be partially due to fibrin having no or little resistance to contraction that occurs naturally as part of the collagen/ECM formation process. The inventors also believe that radial and/or longitudinal contraction occurs in part naturally as an inherent function of tissue forming as described herein. In another embodiment of the invention, the contraction may be scalable or intentionally controlled to enhance, promote, or achieve one or more tissue characteristics, e.g., fiber alignment, or tensile strength, or suturability. Furthermore, the CT of the present invention does not include any synthetic materials, as is typical in other processes that use PLA, PGA, or the like.

The CT of the present invention may be characterized by one or more of the following: non-oriented fibers; oriented fibers; thickness up to about 2 mm, preferably between about 100 μm and about 800 μm; diameters greater than about 1 mm; diameters from about 1 mm to about 40 mm, preferably from about 2 mm to about 25 mm, most preferably from about 3 mm to about 16 mm; lengths greater than about 1 cm; lengths from about 1 cm to about 100 cm, preferably 10 cm to 30 cm, and most preferably about 12 cm to about 22 cm; non-immunogenic or minimally immunogenic; a tissue, sheet or shape that is anisotropic; a tissue, sheet, or shaped structure produced by a process that includes scaled contraction (as described above); a sheet or shape that is suitable for cutting into shapes, e.g., by scalpel, die, or laser; suppleness; suturability; no or little calcification during life of implant; crosslink density, or variations of crosslink density through the material thickness; collagen concentration; collagen density, or variation of crosslink density through the material thickness; remodeling proclivity; absorption; resorption; degradability, regions of greater stiffness; regions of greater flexibility.

In some embodiments of the invention, the diameter and diameter range may be determined by the type of graft or implant being used. The diameter may be any diameter, and typically less than about 6 mm (but it can also be greater). For example, a CABG graft preferably has a diameter from about 3 mm to about 6 mm.

In some embodiments of the invention, the length and length range may be determined by the type of graft or implant being used. The length may be any length. For example, a CABG product preferably has a length from about 15 cm to about 25 cm, which is then sized as needed during the implant procedure, typically from about 8 cm to about 15 cm.

In accordance with some embodiments of the invention, either or both of the diameter and the length can be tapered.

In some embodiments of the invention, the thickness and thickness range may be determined by the type of graft or implant being used. For example, a CABG graft preferably has a thickness from about 0.3 mm to about 0.8 mm; an AV access graft may have a thickness from about 0.3 mm to about 1.0 mm.

The tissue or a product of the present invention may be used to treat or repair other types of tissues including but not limited to muscle, tendon, organs (e.g., kidneys and liver) skin, vasculature, bladder, fascia, and uterus.

Now referring to the FIGURES, the FIGURE is a Kaplan Meier plot of primary and secondary patency of a graft of the present invention.

### Various Method Steps:

By mimicking the extra cellular matrix of the natural environment, a tissue are grown having desirable or beneficial structural properties, which eventually develop toward a native-like architecture (i.e., the tissues of the present invention are a biomimetic material). In preferred embodiments of the invention, the tissue of the present invention may be handled in a similar way like a native vein or artery when surgically implanted.

Some embodiments of the invention may further include storing and/or sterilizing a medical device or tissue of the present invention. These embodiments may include preselected storage solution; preselected sterilization solution or technique; storage packaging; and/or sterilization packaging. In one embodiment, the tissue may be stored in PBS and refrigerated until use. In another embodiment, the tissue may be partially or fully dehydrated. In one embodiment, the storage is in a sterile dry container. Other storage/sterilization processes may include one or more additives known to those with skill in the art. In another embodiment, the tissue may be E-beam sterilized in PBS alone.

One skilled in the art will recognize that other storage and sterilization protocols may be used with the tissue of the present invention.

In some embodiments of the invention, the tissue when implanted in humans does not degrade prior to recellularization or body cell infiltration.

### OTHER EMBODIMENTS

A tissue of the present invention may also define or enclose a total volume from about 3 mm³ to about 65,000 mm³.

The tissue of the present invention may include one or more collagens, including but not limited to collagen types I, III, and VI; tenascin, and fibronectin.

As noted above, a tissue product of the present invention is a tube or conduit, or tubular in shape. A preferred embodiment is a continuous tubular shape. Typically, these shapes include or enclose a lumen through the longitudinal length of the construct. The lumen can be open or closed at one or both ends, and may be shaped at one or both ends.

In some embodiments of the invention, the implanted tissue, graft, or implant is capable of endothelialization, even substantial endothelialization, a feature that provides evidence of the long-term biocompatibility of the tissue and mediation in vascular repair.

In various embodiments of the invention, including but not limited to CABG, pre-clinical studies showed exemplary hemodynamics and biomimetic function. All of these evaluations showed that the CT tissue of the present invention has similar compliance and “hand” to native structures.

In preferred embodiments of the invention, CT tissue grafts have been shown to provide anatomical and functional characteristics that mimic native structures, e.g., native vessels around the heart and saphenous veins.

In some embodiments of the invention, the CABG is a sterile, ~4-6 mm inner diameter, ~15 cm long tubular conduit composed of a naturally produced acellular human collagen matrix. The collagen matrix is produced by culturing human dermal fibroblast (HDF) cells in a controlled process. The product possesses structural integrity with burst strength and suture retention strengths similar to native arteries and veins (e.g., 3000-5000 mmHg burst strength).

The present invention also is a surgical kit comprising one or more of the following: a regenerative tissue implant or graft processed or produced according to the present invention; one or more instruments for implanting the graft; a rinse tray; a rinse solution, e.g., heparin; and suture material.

One of the embodiments of the present invention is a sterile closed package containing a biological material of the present invention and an external support structure configured to slide over the biological material. Typically, a separate container may hold individual or multiple samples having known size or dimensions.

The present invention also includes tubular grafts in a variety of diameters and lengths as needed to match the anatomy.

An implant of present invention maybe delivered in any medically acceptable manner. In a preferred embodiment, the tissue is surgically delivered. In another embodiment, the tissue is delivered via catheter or tube.

One skilled in the art will recognize that the processes steps described herein may be variously modified and a wide variety of ways in order to achieve a tissue with certain properties, according to present invention.

### Definitions

The following definitions are used in reference to the invention:

As indicated herein, a decellularized vessel consists essentially of the extracellular matrix (ECM) components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures.

(B) As used herein, biomimetics or biomimicry refer to imitating the models, systems, and elements of nature for the purpose of solving complex human or animal problems. In the present invention, biomimetics is used for therapeutic purposes.

### REFERENCES

1. P. A. Gurbel, K. A. A. Fox, U. S. Tantry, H. Ten Cate, J. I. Weitz, Combination Antiplatelet and Oral Anticoagulant Therapy in Patients With Coronary and Peripheral Artery Disease. Circulation 139, 2170-2185 (2019).
2. M. Desai, A. M. Seifalian, G. Hamilton, Role of prosthetic conduits in coronary artery bypass grafting. Eur J Cardiothorac Surg 40, 394-398 (2011).
3. S. Pashneh-Tala, S. MacNeil, F. Claeyssens, The Tissue-Engineered Vascular Graft-Past, Present, and Future. Tissue Eng Part B Rev 22, 68-100 (2016).
4. L. Englberger et al., The Shelhigh No-React bovine internal mammary artery: a questionable alternative conduit in coronary bypass surgery? Eur J Cardiothorac Surg 33, 222-224 (2008).
5. F. E. M. Herrmann et al., Autologous endothelialized vein allografts in coronary artery bypass surgery-Long term results. Biomaterials 212, 87-97 (2019).
6. T. Hasegawa et al., Hybrid small-caliber vascular prosthesis for coronary artery bypass grafting: a preliminary study of plasmin-treated fibrin-coated vascular prosthesis. ASAIO J 51, 725-729 (2005).
7. Z. H. Syedain et al., A completely biological “off-the-shelf” arteriovenous graft that recellularizes in baboons. Sci Transl Med 9, (2017).
8. S. L. Dahl et al., Readily available tissue-engineered vascular grafts. Sci Transl Med 3, 68ra69 (2011).
9. A. L. Lauria et al., Preliminary Experience With the Human Acellular Vessel: A Descriptive Case Series Detailing Early Use of a Bioengineered Blood Vessel for Arterial Repair. Ann Vasc Surg 87, 100-112 (2022).
10. P. Gutowski et al., Arterial reconstruction with human bioengineered acellular blood vessels in patients with peripheral arterial disease. J Vasc Surg 72, 1247-1258 (2020).
11. J. J. Morrison et al., Clinical implementation of the Humacyte human acellular vessel: Implications for military and civilian trauma care. J Trauma Acute Care Surg 87, S44-S47 (2019).
12. S. G. Adrian Ebner, Juan Castellano, Maria Filartiga, Allie Haarstad, Cory Pries, Kate Gahlbeck, Mariah McMahon, Camila Herrera, Martha Avila, Kem Schankereli, Rick Murphy, Mark Stenoien, Zeeshan Syedain, Haimanot Wasse, First-in-human evaluation of a biological regenerative vascular conduit for hemodialysis access. The Journal of Vascular Access In Press, (2023).
13. J. H. Lawson et al., Bioengineered human acellular vessels for dialysis access in patients with end-stage renal disease: two phase 2 single-arm trials. Lancet 387, 2026-2034 (2016).
14. L. C. Bylsma et al., Clinical Outcomes of Arteriovenous Access in Incident Hemodialysis Patients with Medicare Coverage, 2012-2014. Am J Nephrol 49, 156-164 (2019).
15. Z. H. Syedain, L. A. Meier, M. T. Lahti, S. L. Johnson, R. T. Tranquillo, Implantation of completely biological engineered grafts following decellularization into the sheep femoral artery. Tissue Eng Part A 20, 1726-1734 (2014).
16. L. A. Meier et al., Blood outgrowth endothelial cells alter remodeling of completely biological engineered grafts implanted into the sheep femoral artery. J Cardiovasc Transl Res 7, 242-249 (2014).
17. M. Ono et al., 1-Year Patency of Biorestorative Polymeric Coronary Artery Bypass Grafts in an Ovine Model. JACC Basic Transl Sci 8, 19-34 (2023).
18. G. Konig et al., Mechanical properties of completely autologous human tissue engineered blood vessels compared to human saphenous vein and mammary artery. Biomaterials 30, 1542-1550 (2009).
19. N. L'Heureux et al., Human tissue-engineered blood vessels for adult arterial revascularization. Nat Med 12, 361-365 (2006).
20. Z. H. Syedain et al., Pediatric tri-tube valved conduits made from fibroblast-produced extracellular matrix evaluated over 52 weeks in growing lambs. Sci Transl Med 13, (2021).
21. M. Stenoein, “Vascudyne, Inc. Investigator Brochure: TRUE HD Study I,” (Vascudyne Inc, 2021).
22. Z. Syedain et al., Tissue engineering of acellular vascular grafts capable of somatic growth in young lambs. Nat Commun 7, 12951 (2016).
23. H. K. Kjaergard, A. Irmukhamedov, J. B. Christensen, T. A. Schmidt, Flow in coronary bypass conduits on-pump and off-pump. Ann Thorac Surg 78, 2054-2056 (2004).
24. Z. Syedain et al., 6-month aortic valve implantation of an off-the-shelf tissue-engineered valve in sheep. Biomaterials 73, 175-184 (2015).
25. J. Reimer et al., Implantation of a Tissue-Engineered Tubular Heart Valve in Growing Lambs. Ann Biomed Eng 45, 439-451 (2017).
26. Z. H. Syedain et al., Tissue-engineered transcatheter vein valve. Biomaterials 216, 119229 (2019).
27. Z. H. Syedain, R. Maciver, R. T. Tranquillo, Vascular grafts and valves that animate, made from decellularized biologically-engineered tissue tubes. J Cardiovasc Surg (Torino) 61, 577-585 (2020).
28. M. T. L. Zeeshan H Syedain, Sandra L. Johnson, Paul S. Robinson, George R. Ruth, Richard W. Bianco, Robert T. Tranquillo, Implantation of a Tissue-engineered Heart Valve from Human Fibroblasts Exhibiting Short Term Function in the Sheep Pulmonary Artery. Cardiovascular Engineering and Technology 2, 101-112 (2011).
29. A. Weigand et al., New aspects on efficient anticoagulation and antiplatelet strategies in sheep. BMC Vet Res 9, 192 (2013).
30. D. J. Goldstein et al., External Support for Saphenous Vein Grafts in Coronary Artery Bypass Surgery: A Randomized Clinical Trial. JAMA Cardiol 7, 808-816 (2022).
31. D. T. Inderbitzin et al., One-year patency control and risk analysis of eSVS (R)-mesh-supported coronary saphenous vein grafts. J Cardiothorac Surg 10, 108 (2015).
32. T. Morota, F. G. Duhaylongsod, W. R. Burfeind, C. T. Huang, Intraoperative evaluation of coronary anastomosis by transit-time ultrasonic flow measurement. Ann Thorac Surg 73, 1446-1450 (2002).
33. N. Tsirikos Karapanos et al., The impact of competitive flow on distal coronary flow and on graft flow during coronary artery bypass surgery. Interact Cardiovasc Thorac Surg 12, 993-997; discussion 997 (2011).
34. L. Hocum Stone et al., Surgical Swine Model of Chronic Cardiac Ischemia Treated by Off-Pump Coronary Artery Bypass Graft Surgery. J Vis Exp, (2018).
35. C. A. Merrifield et al., A metabolic system-wide characterization of the pig: a model for human physiology. Mol Biosyst 7, 2577-2588 (2011).
36. L. Van Hoof et al., Apixaban in a porcine model of mechanical valve thrombosis in pulmonary position-a pilot study. Interact Cardiovasc Thorac Surg 35, (2022).
37. M. P. Bonaca et al., Long-term use of ticagrelor in patients with prior myocardial infarction. N Engl J Med 372, 1791-1800 (2015).
38. C. S. Committee, A randomised, blinded, trial of clopidogrel versus aspirin in patients at risk of ischaemic events (CAPRIE). CAPRIE Steering Committee. Lancet 348, 1329-1339 (1996).
39. D. L. Bhatt et al., Clopidogrel and aspirin versus aspirin alone for the prevention of atherothrombotic events. N Engl J Med 354, 1706-1717 (2006).
40. J. W. Eikelboom et al., Rivaroxaban with or without Aspirin in Stable Cardiovascular Disease. N Engl J Med 377, 1319-1330 (2017).
41. S. J. Connolly et al., Rivaroxaban with or without aspirin in patients with stable coronary artery disease: an international, randomised, double-blind, placebo-controlled trial. Lancet 391, 205-218 (2018).
42. E. M. Ohman et al., Clinically significant bleeding with low-dose rivaroxaban versus aspirin, in addition to P2Y12 inhibition, in acute coronary syndromes (GEMINI-ACS-1): a double-blind, multicentre, randomised trial. Lancet 389, 1799-1808 (2017).
43. A. Avezum et al., Apixaban in Comparison With Warfarin in Patients With Atrial Fibrillation and Valvular Heart Disease: Findings From the Apixaban for Reduction in Stroke and Other Thromboembolic Events in Atrial Fibrillation (ARISTOTLE) Trial. Circulation 132, 624-632 (2015).
44. Y. Torres et al., Cell-assembled extracellular matrix (CAM) sheet production: Translation from using human to large animal cells. J Tissue Eng 12, 2041731420978327 (2021).
45. T. N. McAllister et al., Effectiveness of haemodialysis access with an autologous tissue-engineered vascular graft: a multicentre cohort study. Lancet 373, 1440-1446 (2009).

### EXAMPLES

### Example 1

A tissue of the present invention was implanted as a CABG graft into an ovine model, and after one year showed long-term performance and regeneration into a living blood vessel.

A tissue of the present invention was implanted as a pediatric vascular conduit into a lamb model, and after one year showed somatic growth and regeneration.

### Example 2

TABLE 1
Human Vessel implant and Comparable Biologically
Engineered Vascular Grafts

| | | | Suture | Burst |
|---|---|---|---|---|
| | Diameter | Thickness | Retention | Pressure |
| Conduit Type | (mm) | (mm) | (gram-Force) | (mmHg)** |
| Present | 4 ± 0.15 | 0.382 ± 0.05 | 370 ± 81 | 2515 ± 305 |
| invention | | | | |
| Xeltis RVG | 4.0 | 0.5 | | |
| (for CABG) | | | | |
| (17) | | | | |
| Human | 1.5-4.5 | 0.3-0.8* | 138 ± 50 | 1599 ± 877 |
| Mammary | | | | |
| artery (18, 19) | | | | |
| Human | 1.5-4.5 | 0.3-0.8* | 196 ± 29 | 3196 ± 1264 |
| saphenous | | | | |
| vein (18, 19) | | | | |

*Only ranges are reported
**probe burst test in place of pressurized burst to decrease sample volume requirement.

### Example 2

Tissue recovered from the animals were fixed in formalin and processed for staining.
H&E-stained sections show small aggregates of inflammatory/immune cells including eosinophils, PMNs, lymphocytes, and macrophages within the abluminal area of the conduits, but rated as absent to minimal/diffuse in all 6-month explants. The 3-month explant (BAVG 7), where they were rated as marked/focal in the abluminal areas of the graft in some sections.
Most trichrome-stained sections showed elongated cells with circumferential orientation in both the 3-month and 6-month explants, indicative of positive remodeling. The conduits all possessed abundant collagen as seen in the trichrome-stained sections.
Fibrin immunohistochemical staining confirmed a thin thrombus layer on the luminal surface of the 3-month explant, also evident in the trichrome staining. Minimal fibrin staining was evident in the 6-month explants.
Verhoeff-van Gieson stained sections revealed mature elastic fibers within the implant near the proximal anastomosis in two of the 6-month explanted conduits, but not seen in other sections.
All conduits at 3 and 6-month stained negative for calcification with Von Kossa stain.
Many cells populating the interstitium of the conduits stained positive for vimentin in all sections for both 3 and 6 months.
The majority of elongated cells in a distinct region adjacent to the luminal surface of many sections stained positive for a-smooth muscle actin (αSMA) at both time points. The thickness of this region, when clearly identifiable, was highly variable but commonly between 100-300 μm.
There was sporadic smoothelin staining of the elongated cells along the base of the aforementioned region.
At 3 months, CD31 staining showed limited endothelium on the distal conduit region with none noted mid-conduit. Whereas the 6-month sections showed complete endothelium in the proximal and distal regions in 2 of 3 patent conduits, and one mid-conduit region, while the remaining sections showed some level of incomplete endothelium coating.
Consistent with the ultrasonography, no stenosis was evident at the distal anastomosis based on histological assessment, with minimal to mild (generally <250 μm in thickness) intimal thickening and fibrosis in the vein at the junction with the implant.

Overall, the immunological analysis shows no adverse acute or chronic immune reaction or sensitization to implanted engineered tissue.

### Example 4

In Gen 1 study, 4 animals survived past 6 months with 3 planned explants accessed for angiography, gross pathology and histopathology to evaluate remodeling of a CABG graft and any adverse outcomes in myocardial tissue. Animals 22S0026, 22S0032 and 22S0034 all had a patent conduit, no abnormal findings in gross pathology and no myocardial tissue damage or evidence of emboli.

The angiogram at 180 days showed uniform diameter transition from a CABG into the native coronary. Further, gross examination showed distal anastomosis without evidence of hyperplastic response. The anastomotic sutures were visible in the 180-day explants. A cross-section of the graft showed remodeled tissue with areas of glossy lumen surface and areas with thin layer of thrombus. Explanted tissue for n=2 conduit was mechanically tested with an average lumen burst strength of 3500±1405 mm Hg, in comparison to implant lumen burst strength of 3109±624 mm Hg, confirming remodeling of the graft without any weakening of the tissue mechanical strength.

Histology of explants at early timepoint confirmed a lack of overt immune response with only few neutrophils migrating at day 8. After 180 days of implant, the implanted graft was fully remodeled with interstitial cells and evidence of endothelial cells in the proximal third of the conduit. While lumen endothelium lacked in the entire length of implant, the interstitial cell remodeling looked similar along the length of implant. Further, remodeling was seen with the graft in coronary implant is comparable to previous reported remodeling with the Baboons after 6 months implant as arteriovenous conduit and in sheep as femoral artery implant.

### Example 5

In the studies of a tissue conduit in the baboon model and the ovine model, all explanted grafts were histologically evaluated for remodeling of the graft tissue. In the ovine model, in explanted grafts at 4 and 8 weeks, an acellular band of original graft was surrounded by remodeled matrix tissue on both luminal and abluminal surfaces. By 6 months, this acellular region had much reduced width suggesting remodeling of the original tissue matrix by invading host cells. The explanted tissue at 180 days was evaluated for mechanical properties and exceeded the properties of implant material, suggesting host tissue formation leading to a stronger graft. As the original matrix and remodeled matrix are both collagenous in nature, no immunostaining is available to differentiate and quantitate the new cell-produced collagen from the implanted collagen.

In the baboon model implanted with tissue grafts, the graft was fully cellularized by 3 months, suggesting complete remodeling of the implanted graft by host cells. This was confirmed by an increase in mechanical burst pressure strength of the explanted graft, which was 136% of implanted grafts. At 6 months, the explanted grafts were also stronger by 156% compared to implanted grafts, including areas with repeated puncture using a 16G dialysis needle. The ECM remodeling is a natural phenomenon and does not include any chemical processes and products that would not occur naturally.

### Example 6

In the preclinical study using sheep and pigs, 4 mm conduits were implanted as coronary artery bypass. The native artery was tied in all cases to prevent competing flow. Majority of implants were done to anterior descending (n=13) with circumflex (n=5) being second and n=3 to diagonal. In the planned explant of the diagonal target at 6 months after implant, evidence of regeneration including endothelium was observed in proximal one-third of the conduit. Subsequent studies in pig with oral anticoagulant (apixaban and aspirin combo) showed no evidence of thrombus on lumen surface up to 30 days after implant.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

While the invention has been described in some detail by way of illustration and example, it should be understood that the invention is susceptible to various modifications and alternative forms and is not restricted to the specific embodiments set forth in the Examples. It should be understood that these specific embodiments are not intended to limit the invention but, on the contrary, the intention is to cover all modifications, equivalents, and alternatives falling within the spirit and scope of the invention.

1. An implant suitable for use in humans comprising a coronary artery bypass conduit engineered or constructed from tissue formed using a composition comprising an extracellular matrix (ECM)-producing cell, thrombin, and fibrinogen; wherein said tissue endothelializes.

2. The implant of claim 1 wherein the tissue is acellular.

3. The implant of claim 1 wherein the implant is about 15 cm long.

4. The implant of claim 1 wherein the conduit is less than about 6 mm in diameter.

5. The implant of claim 4 wherein the conduit is about 4 mm to about 6 mm in diameter.

6. The implant of claim 4 wherein the conduit is about 4 mm in diameter.

7. The implant of claim 4 wherein the conduit is about 6 mm in diameter.

8. The implant of claim 3 further comprising wherein the implant is tapered.

9. The implant of claim 4 further comprising wherein the implant is tapered.

10. The implant of claim 1 wherein the tissue induces recipient cellular regeneration or remodeling at or near the site of implant.

11. The implant of claim 1 wherein the tissue contracts during engineering or construction by less than about 50%.

12. The implant of claim 1 wherein the tissue or the implant is biomimetic.

13. The implant of claim 1 wherein the implant is a pediatric bypass.

14. The implant of claim 1 wherein the tissue comprises non-uniform layers of collagen.

15. The implant of claim 1 wherein said tissue is isotropic.

16. The implant of claim 1 wherein said tissue induces in vivo recellularization without degradation.
